(19) United States
(12) Patent Application Publication
(10) Pub. No.: US 2024/0307678 A1
(43) Pub. Date: Sep. 19, 2024

(54) BIODEGRADABLE LEADS AND SYSTEMS INCLUDING BIODEGRADABLE LEADS

(71) Applicant: University of Pittsburgh - Of the Commonwealth System of Higher Education, Pittsburgh, PA (US)

(72) Inventors: Xinyan Cui, Wexford, PA (US); Trent D. Emerick, Mars, PA (US); Kevin M. Woeppel, Pittsburgh, PA (US); Rajkumar Chinnakonda Kubendran, Pittsburgh, PA (US)

(21) Appl. No.: 18/264,999

(22) PCT Filed: Feb. 15, 2022
(86) PCT No.: PCT/US2022/016390

§ 371 Date: Aug. 10, 2023

Related U.S. Application Data
(60) Provisional application No. 63/149,496, filed on Feb. 15, 2021.

(51) Int. Cl. A61N 1/05; A61N 1/36

(52) U.S. Cl. CPC A61N 1/0551; A61N 1/36071

(57) ABSTRACT

An implantable system includes at least one implantable lead including a biodegradable conductive core, a biodegradable polymeric insulator encompassing at least a portion of a length of the biodegradable conductive core, and at least one electrode on a distal end thereof. The implantable system further includes electronic circuitry operatively connectible to the at least one implantable lead which is configured to provide a controlled electrical signal via the at least one electrode of the at least one implantable lead to tissue to effect treatment (for example, pain treatment).

### CROSS-REFERENCE TO RELATED APPLICATIONS

This application claims benefit of U.S. Provisional Patent Application Ser. No. 63/149,496, filed Feb. 15, 2021, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

It has been estimated that 11.2% of the U.S. adult population suffers from daily chronic pain. Severe pain is associated with worse health and a greater level of disability, and it results in a significant annual economic burden. Unfortunately, pharmaceutical treatments can lead to substance abuse and overdose, which has fueled a devastating opioid epidemic. Therefore, alternative pain treatments such as neurostimulation therapy via spinal cord stimulation (SCS) and peripheral nerve stimulation (PNS) are often utilized.

In that regard, patients in severe refractory and chronic pain often elect to have one or more stimulator leads, wires or electrodes (for example, titanium or platinum stimulator leads) placed along the spinal cord or various peripheral nerves to help with pain control. Upon implanting one or more electrodes near a nerve or the spinal cord, an electrical stimulus may be applied via an implanted pulse generator to modulate the perception of pain. The leads, wires or electrode leads and associated electrodes operatively connected to the implanted pulse generator can modulate pain and improve physical function. Nearly 100,000 patients receive stimulator surgical implants per year. Most are in excess of 50 years old. Long term pain relief is often achieved even after the cessation of stimulation, and chronic stimulation is not always required to treat chronic pain. The result is an analgesic effect without the use of addictive drugs. Furthermore, SCS and PNS can be used to treat neuropathic and nociplastic pain, which are resistant to opioids.

Methods and devices for implanting of stimulating leads, lead location, and applying various stimulation waveforms are, for example, described in PCT International Patent Application Publication Nos. 2013/036620 and WO2017/142948, US Patent Application Publication No. 2017/0340891, Australian Patent No. AU2017221321 B2, and U.S. Pat. Nos. 10,056,688 and 10,245,435, the disclosures of which are incorporated herein by reference.

Patients with chronic pain who have failed conservative therapies such as physical therapy, injections, and medication management lack alternative treatment options other than the surgical implantation of a permanent nerve stimulator device. As described above, such stimulator devices may include titanium or platinum leads (that is, insulated electrical conductors or wires) and a battery pack/controller that are surgically inserted and cannot be removed without additional surgery. However, as many as 30 to 40% of patients with stimulator device implants have complications such as lead migration, lead fractures or disconnections, or development of new pain sites after lead implantation. Permanently implanted wires can also become a source of chronic pain and/or other problems over time. Patients with implanted stimulator leads are left with an implantable device that may have MRI compatibility issues, infection risks, and security screening restrictions. In many cases, such complications require a revision surgery or surgical explantation of the leads.

Furthermore, a portion of patients that have a stimulator device in place may no longer use the device for a variety of the reasons. In some cases, the pain resolves or the quality of pain changes, and the stimulator leads are no longer helpful or are no longer needed. These patients are often hesitant to have another surgery to remove the leads.

Efforts have been made to address these limitations, including the development of minimally invasive and temporary devices. Percutaneous PNS electrodes that can be delivered through non-surgical injection procedures have been reported. Although such a percutaneous design minimizes the invasiveness of implantation and can be used for short-term treatment, the device still needs to be explanted in the case of undesired events (for example, lead fracture, infection, or noxious sensations). A temporary PNS electrode is approved for human use in the US for 60 days but must be removed by a physician afterwards. Lead removal in connection with that device is associated with a 15% risk of lead fracture resulting lead fragments left inside the body. Such retained lead fragments lead to further limitations in, for example, future MRIs and unknown effects on local neurovascular and musculoskeletal structures.

There remains a need to develop alternative devises, systems, methods, and compositions to address problems associated with currently available nerve stimulation technologies in, for example, the treatment of chronic pain.

### SUMMARY

In one aspect, an implantable system includes at least one implantable lead including a biodegradable conductive core, a biodegradable polymeric insulator encompassing at least a portion of a length of the biodegradable conductive core, and at least one electrode on a distal end thereof. The implantable system further includes electronic circuitry operatively connectible to the at least one implantable lead which is configured to provide a controlled electrical signal via the at least one electrode of the at least one implantable lead to tissue to effect treatment (for example, pain treatment, restoration of motor function, etc.). The controlled electrical signal may include pulses of electrical energy. The electronic circuitry may be configured to be positioned ex vivo when placed in operative connection with the at least one implantable lead and the implantable lead is configured to be implanted percutaneously.

In a number of embodiments, the biodegradable conductive core includes at least one of a biodegradable metal, a biodegradable metal alloy or a biodegradable conductive polymer. In a number of embodiments, the biodegradable conductive core includes a biodegradable metal or a biodegradable metal alloy. The biodegradable metal or biodegradable metal alloy may, for example, include magnesium, a magnesium alloy, iron, an iron alloy, zinc, a zinc alloy, molybdenum, a molybdenum alloy, zirconium, a zirconium alloy, calcium, or a calcium alloy. Magnesium alloys may, for example, include at least one of zinc, aluminum, copper, cerium, calcium, silver, thorium, gadolinium, dysprosium, strontium, silicon, manganese, zirconium, neodymium or yttrium. In a number of embodiments, the biodegradable conductive core includes zinc or an alloy of zinc. Zine alloys may, for example, include at least one of magnesium, lithium, copper, iron, manganese, silver, calcium, strontium, zirconium, sodium, potassium, chromium, yttrium, tin, aluminum, barium, bismuth, and germanium. In a number of embodiments, the biodegradable conductive core includes zinc. In a number of embodiments, the biodegradable polymeric insulator includes a biodegradable polyurethane polymer or copolymer or a polyurethane urea polymer or copolymer.

The composition and/or physiochemical properties of the biodegradable conductive core may be formulated to provide a predetermined degradation profile over time. Likewise, the composition and/or physiochemical properties of the biodegradable polymeric insulator may be formulated to provide a predetermined degradation profile over time.

In a number of embodiments, the system includes a plurality of implantable biodegradable leads. At least one of plurality of implantable biodegradable leads may, for example, function as a recording electrode and the electronic circuitry is configured to adjust the controlled electrical signal on the basis of feedback information from the recording electrode.

In a number of embodiments, the electronic circuitry is further configured to measure impedance at the interface of the tissue and the at least one implantable lead via which the controlled electrical signal is provided and to adjust the controlled electrical signal on the basis of the measured impedance. In a number of embodiments, the electronic circuitry is configured to transmit a degradation acceleration electrical signal to the least one implantable lead to increase a rate of degradation thereof. The degradation acceleration electrical signal may, for example, convert the biodegradable conductive core to a form more readily resorbed in vivo. In embodiments in which the biodegradable conductive core includes a metal or a metal alloy, the degradation acceleration electrical signal may oxidize the metal or the metal alloy.

In another aspect, a method of transmitting electrical signals to in vivo tissue for treatment includes implanting at least one implantable lead which includes a biodegradable conductive core, a biodegradable polymeric insulator encompassing at least a portion of a length of the biodegradable conductive core, and at least one electrode on a distal end thereof, and applying a controlled electrical signal to the tissue via the at least one electrode of the at least one implantable lead via electronic circuitry operatively connected to the at least one implantable lead. The at least one implantable lead may, for example, be implanted via injection (for example, via a hollow-bore needle). In a number of embodiments, the at least one implantable lead is used to transmit the electrical signal for pain therapy electrical stimulation.

The controlled electrical signal may include pulses of electrical energy. The electronic circuitry may be configured to be positioned ex vivo when placed in operative connection with the at least one implantable lead and the implantable lead is configured to be implanted percutaneously.

In a number of embodiments, the biodegradable conductive core includes at least one of a biodegradable metal, a biodegradable metal alloy or a biodegradable conductive polymer. In a number of embodiments, the biodegradable conductive core includes a biodegradable metal or a biodegradable metal alloy. The biodegradable metal or biodegradable metal alloy may, for example, include magnesium, a magnesium alloy, iron, an iron alloy, zinc, a zinc alloy, molybdenum, a molybdenum alloy, zirconium, a zirconium alloy, calcium, or a calcium alloy. Magnesium alloys may, for example, include at least one of zinc, aluminum, copper, cerium, calcium, silver, thorium, gadolinium, dysprosium, strontium, silicon, manganese, zirconium, neodymium or yttrium. In a number of embodiments, the biodegradable conductive core includes zinc or an alloy of zinc. Zinc alloys may, for example, include at least one of magnesium, lithium, copper, iron, manganese, silver, calcium, strontium, zirconium, sodium, potassium, chromium, yttrium, tin, aluminum, barium, bismuth, and germanium. In a number of embodiments, the biodegradable conductive core includes zinc. In a number of embodiments, the biodegradable polymeric insulator includes a biodegradable polyurethane polymer or copolymer or a polyurethane urea polymer or copolymer.

The composition and/or physiochemical properties of the biodegradable conductive core may be formulated to provide a predetermined degradation profile over time. Likewise, the composition and/or physiochemical properties of the biodegradable polymeric insulator may be formulated to provide a predetermined degradation profile over time.

In a number of embodiments, the system includes a plurality of implantable biodegradable leads. At least one of plurality of implantable biodegradable leads may, for example, function as a recording electrode and the electronic circuitry is configured to adjust the controlled electrical signal on the basis of feedback information from the recording electrode.

In a number of embodiments, the method further includes measuring impedance at the interface of the tissue and the at least one implantable lead via which the controlled electrical signal is provided via the electronic circuitry and adjusting the controlled electrical signal via the electronic circuitry on the basis of the measured impedance. In a number of embodiments, the method further includes transmitting a degradation acceleration electrical signal to the least one implantable lead via the electronic circuitry to increase a rate of degradation thereof. The degradation acceleration electrical signal may, for example, convert the biodegradable conductive core to a form more readily resorbed in vivo. In embodiments in which the biodegradable conductive core includes a metal or a metal alloy, the degradation acceleration electrical signal may oxidize the metal or the metal alloy.

In another aspect, an implantable lead hereof consists essentially of a biodegradable conductive core formed of a metal or a metal alloy, and a biodegradable polymeric insulator directly adjacent to and encompassing at least a portion of a length of the biodegradable conductive core. The biodegradable metal or the metal alloy may, for example, include magnesium, a magnesium alloy, iron, an iron alloy, zinc, a zinc alloy, molybdenum or a molybdenum alloy, zirconium, a zirconium alloy, calcium, or a calcium alloy. In a number of embodiment, the metal or the metal alloy includes zine or a zinc alloy. Zine alloys may, for example, include at least one of magnesium, lithium, copper, iron, manganese, silver, calcium, strontium, zirconium, sodium, potassium, chromium, yttrium, tin, aluminum, barium, bismuth, and germanium. In a number of embodiments, the metal is zinc. A magnesium alloy for use in implantable leads hereof may, for example, include at least one of zinc, aluminum, copper, cerium, calcium, silver, thorium, gadolinium, dysprosium, strontium, silicon, manganese, zirconium, neodymium or yttrium. The biodegradable polymeric insulator may, for example, include a biodegradable polyurethane or a polyurethane urea polymer or copolymer.

In a number of embodiments, the composition and/or physiochemical properties of the biodegradable conductive core are formulated to provide a predetermined degradation profile over time. In a number of embodiments, the composition and/or physiochemical properties of the biodegradable polymeric insulator are formulated to provide a predetermined degradation profile over time.

In a further aspect, a control system for use with at least one biodegradable implantable lead includes electronic circuitry operatively connectible to the at least one biodegradable implantable lead wherein the electronic circuitry is configured to provide a controlled electrical signal to tissue via the at least one implantable lead to effect treatment, and wherein the electronic circuitry is further configured to measure impedance at an interface of the at least one implantable lead and the tissue and to adjust the controlled electrical signal on the basis of the measured impedance. In a number of embodiments, the electronic circuitry is further configured to transmit a degradation acceleration electrical signal to the least one biodegradable implantable lead to increase a rate of degradation thereof. The degradation acceleration electrical signal may, for example, convert the biodegradable conductive core to a form more readily resorbed in vivo. In a number of embodiments in which the biodegradable conductive core includes a metal or a metal alloy, and the degradation acceleration electrical signal oxidizes the metal or the metal alloy.

In still a further aspect, a control system for use with at least one biodegradable implantable lead includes electronic circuitry operatively connectible to the at least one biodegradable implantable lead which is configured to provide a controlled electrical signal to tissue via the at least one implantable lead to effect treatment and wherein the electronic circuitry is configured to transmit a degradation acceleration electrical signal to the least one biodegradable implantable lead to increase a rate of degradation thereof. In a number of embodiments, the electronic circuitry is further configured to measure impedance at an interface of the tissue and the at least one implantable lead and to adjust the controlled electrical signal on the basis of the measured impedance.

As described above, the degradation acceleration electrical signal may, for example, convert the biodegradable conductive core to a form more readily resorbed in vivo. In a number of embodiments, the biodegradable conductive core includes a metal or a metal alloy, and the degradation acceleration electrical signal oxidizes the metal or the metal alloy.

The present devices, systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates an embodiment of a device or system hereof including a biodegradable conductive lead implanted via ultrasound guidance to interact with the ulnar nerve.

FIG. 2 illustrates the conductivity and stiffness of the biodegradable metals magnesium (Mg) and zinc (Zn) versus stainless steel and platinum.

FIG. 3 illustrates the degradation chemistry of magnesium and zinc and sets forth a number of therapeutic effect of Mg²⁺ and Zn²⁺.

FIG. 4 illustrates alteration or tuning of the degradation rate of magnesium via, for example, alloying the magnesium with aluminum (Al) and zinc (Zn).

FIG. 5A illustrates degradation studies of an isophorone-based polyurethane polymer over time for four layers of polymer and eight layers of polymer on silver (Ag) wire wherein impedance at 1 kHz is set forth over time.

FIG. 5B illustrates degradation studies of isophorone-based polyurethane polymer and hexamethylene-based polyurethan over time for four layers of polymer on silver (Ag) wire wherein impedance at 1 kHz is set forth over time.

FIG. 5C illustrates an embodiment of a synthetic scheme for a biodegradable segment or linker (a macrodiol) for biodegradable polyurethanes hereof wherein a copolymer of caprolactone and valerolactone is formed via the addition of diethylene glycol in the presence of a catalyst.

FIG. 5D illustrates an embodiment of a synthetic scheme for the studied isophorone-based polyurethane where isophorone diisocyanate is first reacted with the degradable linker in a catalytic amount of Sn(Oct)₂ and a chain extender (1,6, diaminohexane) is added to the mixture.

FIG. 5E illustrates an embodiment of a synthetic scheme for the studied hexamethylene-based polyurethane where hexamethylene diisocyanate is first reacted with the degradable linker in a catalytic amount of Sn(Oct)₂ and a chain extender (1,6, diaminohexane) is added to the mixture.

FIG. 6 illustrates an in vitro testing system to, for example, study electrical functionality and degradation of biodegradable conductive leads hereof.

FIG. 7 illustrates stability studies of the Zn electrode during stimulation and rapid or enhanced degradation under a degradation on command (DOC) via anodic pulses: (A) Schematic representation of tested embodiment of prototype device. (B) Graph of voltage excursions from a 20 us-20 mA pulse followed by a 40 μs 10 mA balancing pulse measured after 1K, 250M and 550M pulses. (C) Graph of impedance measured from Zn electrodes under the same conditions as (B). (D-E) SEM images of pristine Zn (D) and Zn electrode after 250M stimulations (E). (F) SEM image of Zn electrode partially degraded by DOC and EDX studies revealing elevated oxygen in the corrosion layer (bottom) and elevated Zn in core (top). (G) Photographs illustrating the progression of Zn degradation with DOC. (H) Graphs of the voltage response of the SS and Zn electrode to anodic pulse train of 1 mA (1 ms on/off).

FIG. 8 illustrates an embodiment of a scheme of synthesis of another representative biodegradable polyurethane used in the studies hereof.

FIG. 9 illustrates characterization of biodegradable polyurethane (BPU) insulation: (A) Graph of the stability of the insulation is controllable by varying the number of coats on a silver wire. Increasing number of coats produces higher quality insulation and slower degradation (B) Graph of an elution assay was performed by soaking samples in media for 1 week and adding the elution media to cultured 313 fibroblast cells. Triton-x was chosen as a positive control. After 24 h, cell viability was measured with XTT assay. (C) Graph of studies wherein 3T3 cells were grown directly on coverslips coated biodegradable polyurethan coating for 48 h.

FIG. 10 illustrates studies of a custom designed pulse generator and validation thereof. A) A photograph of a breadboard embodiment of a custom stimulator and field programmable gate array. B) A photograph of a rat paw flexion while stimulating. C) A graph of peak EMG signals show increased muscle activity with increasing stimulus amplitude. * p<0.05 ** p<0.01.

FIG. 11A illustrates a block diagram of an embodiment of an external pulse generator backpack with battery, reference clock, and serial peripheral interface (SPI interface).

FIG. 11B illustrates a block diagram of integrated circuit (IC) architecture for an embodiment of a pulse generator custom ASIC hereof.

FIG. 12 illustrates Table 1 hereof which provides representative specification for a pulse generator hereof.

FIG. 13 illustrates the results of animal studies with Zn electrodes: A) A graph of muscle activation threshold over time which was found to be steady over 4 weeks. B) A graph of impedance recorded over the 4-week implantation. C) A photomicrograph of post-mortem hematoxylin and eosin (H&E) staining of the stimulated nerve.

FIG. 14 illustrates an in vivo DOC study. A) Photograph of a dissected tissue showing a segment of sciatic nerve, the original implant location. The white dashed line portion indicates the location of the uninsulated electrode portion which has completely degraded upon 200 min of DOC, while the insulated portion remain visible thanks to the polyurethane coating. The nerve near DOC appear healthy. B) Photomicrograph of H&E staining of a cross-section of the nerve shows morphology no different from a photomicrograph of contralateral no-DOC control. C) Photomicrograph of H&E staining of contralateral no-DOC control.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a lead” includes a plurality of such lead and equivalents thereof known to those skilled in the art, and so forth, and reference to “the lead” is a reference to one or more such leads and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

As used herein, the term “biodegradable” refers generally to the ability of the material to be broken down (especially into innocuous degradation products) over time in the environment of use (for example, within the body). As used herein, the term “biocompatible” refers generally to compatibility with living tissue or a living system. In that regard, the degradation products of the biodegradable leads thereof may be biocompatible, substantially nontoxic and/or substantially non-injurious to the living tissue or living system in the amounts present over the period of contact/exposure. Moreover, such materials preferably do not cause a substantial negative immunological reaction or rejection in the amounts required over the period of contact/exposure. As used herein, the term “nontoxic” generally refers to substances which, upon ingestion, inhalation, or absorption through the skin by a human or animal, do not cause, either acutely or chronically, damage to living tissue, impairment of the central nervous system, severe illness or death. The term “nontoxic” as used herein may be understood in a relative sense and includes, for example, substances that have been approved by the United States Food and Drug Administration (“FDA”) for administration to or use in humans or, in keeping with established regulatory criteria and practice, are susceptible to approval by the FDA for administration to or use in humans. In addition to FDA approval or approvability, nontoxicity of compounds suitable for use herein can generally be evidenced by a high LD50 as determined in animal studies (for example, rat studies). A nontoxic compound may, for example, have an LD50 of over 1000 mg/kg.

As used herein, the term “polymer” refers to a compound having multiple repeat units (or monomer units) and includes the term “oligomer,” which is a polymer that has only a few repeat units. The term “copolymer” refers to a polymer including two or more dissimilar repeat units (including terpolymers-including three dissimilar repeat units-etc.).

The terms “electronic circuitry”, “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

The term “lead” typically refers to a stimulation device or apparatus from its distal anchor in tissue all the way to its proximal connection to a stimulus generating unit, including portions that may be coated by a biodegradable insulating material hereof. Typically, the term “electrode” refers to the exposed, electrically conductive portion of the lead that is positioned within the body to deliver stimulation. Peripheral nerve stimulator leads traditionally have only a single active non-insulated portion at the distal tip of the lead. However, spinal cord stimulator leads often have a plurality of electrodes (for example, 8-16 different electrodes) per lead. Biodegradable/bioresorbable leads hereof may include multiple electrodes (for example, anodes or cathodes).

As described above, a significant gap in patient care exists in the area of tissue (for example, nerve) stimulation in the treatment of, for example, a medical condition such as chronic pain. In a number of embodiments, devices, systems, methods, and compositions hereof fill that gap through implantation of one or more temporary, biodegradable stimulator leads in operative connection with ex vivo electronic circuitry for delivery of a stimulative electric signal for treatment. The conductive lead(s), wire(s), or electrode lead(s) hereof may, for example, be implanted via a hollow-bore needle under external guidance and do not require a surgery for implantation (in that regard, no skin or tissue incision is required, only needle penetration). Moreover, no sedation or anesthesia is required for the procedure. Examples of hollow bore needles suitable for use to implant biodegradable leads hereof include Tuohy needles (a hollow bore needle often used in inserting epidural catheters) or the PAJUNK® Echogenic catheter-through-needle system available from PAJUNK Medical Systems LP of Norcross, Georgia USA. The biodegradable conductive lead(s) hereof will last for a period of time (for example, a month or longer) to help modulate pain and/or “reset” the nerve(s) to a non-pain state. The rate of degradation of the leads hereof and/or individual components thereof may be controlled through, for example, control of composition, construction, and/or applied electrical signals as described further below. To the contrary, currently available leads used in tissue/nerve stimulation in treatment protocols are formed from a metal such as titanium and require a surgery for permanent placement or for removal.

The biodegradable leads hereof may be used in any system and/or methodology in which an implantable, insulated, biodegradable conductor is desirable (for example, to transmit or to provide an electrical signal). A number of representative embodiments hereof are discussed in connection with nerve stimulation. The biodegradable leads hereof may, for example, be used for peripheral nerve stimulation as well as for spinal cord stimulation (for example, by placing leads in the epidural space to stimulate the dorsal column and dorsal horn of the spinal cord). The biodegradable leads hereof may also be used in muscle stimulation, in brain stimulation (for example, motor cortex stimulation), and in electrically controlled drug delivery devices, systems, and methods. Examples of specific therapeutic uses for stimulation include spinal cord stimulation in the epidural space, temporary spinal cord stimulation in the epidural space for the required trial prior to a permanent implant surgery, dorsal root ganglion stimulation, ganglion stimulation, bladder stimulation, cranial nerve stimulation (including the vagus nerve), ulnar nerve stimulation, peripheral field stimulation in tissues such as muscle, post-operative acute pain nerve stimulation, and brain stimulation for movement disorders or refractory mood disorders. The fully bioresorbable and injectable leads hereof may, for example, be used in connection with representative target indications such as regional pain syndrome (for example, International Classification of Diseases ICD-10 diagnosis code G90.5) and diabetic peripheral neuropathy (ICD-10 diagnosis code E11.42). Such indications use a pre-existing non-experimental, Medicare reimbursement code, CPT 64555, that are already used by other peripheral nerve stimulator systems. Spinal cord stimulation (and peripheral nerve stimulation) with biodegradable lead hereof can also be used in treatments for restoration of motor function through selective stimulation of motor fibers or motor tracts. See, for example, Minassian, K. et al., Spinal Cord Stimulation and Augmentative Control Strategies for Leg Movement after Spinal Paralysis in Humans, CNS Neuroscience & Therapeutics, 22, 262-270 (2016).

In a number of embodiments, biodegradable leads hereof include a biodegradable, conductive and extending core including, for example, at least one of a biodegradable metal, a biodegradable metal alloy, and a biodegradable conducting polymer. A biodegradable insulating polymer may be provided around the biodegradable conductive core as a surrounding insulator. The stimulation capability and degradation profile of the biodegradable leads hereof may be tuned or predetermined for a given application. Properties for materials used in the leads hereof may be readily determined from literature and via in vitro and/or in vivo studies/testing as described herein (for example, in simulated body fluid and/or in animal implantation and testing). Such leads may, for example, be used in systems hereof to provide medium-term or long-term treatment (for example, pain relief for both acute perioperative neuropathic pain and chronic neuropathic pain states).

Conductive, biodegradable stimulator leads (which may also be referred to as elements, electrode leads, or wires) hereof differ significantly from currently available technologies. The biodegradable nature of the leads diminishes or eliminates the need for surgical explantation or revision surgeries. In many cases, a temporary, biodegradable lead hereof can modulate pain and provide mid-term to long-term relief at least as well as currently available permanently implantable leads.

Many patients with chronic nerve pain have often failed a number of conservative therapies and are required to live with debilitating symptoms. Such patients are often not interested in further surgery such as required with a permanent surgical stimulator implant. A biodegradable nerve stimulator implanted under, for example, ultrasound guidance provides a minimally-invasive, non-surgical option that is not currently available. The degradable leads hereof may, for example, be readily fabricated to be sufficiently small for an ultrasound (and/or other imaging/locating system) guided injection, which significantly reduces invasiveness and eliminates the need for surgery.

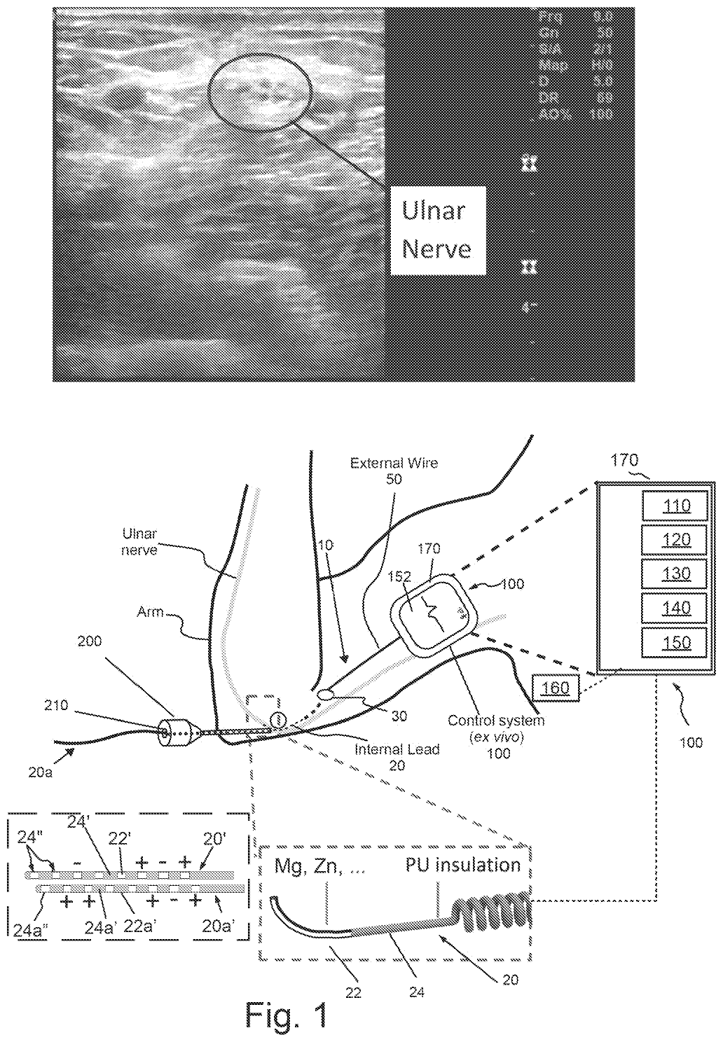

FIG. 1 illustrates an embodiment of a system 10 hereof in which an internally implantable biodegradable lead 20 is implanted to stimulate the ulnar nerve. Biodegradable lead 20 may, for example, be implanted via a bore 210 of a hollow bore needle 200 under ultrasonic and/or other imaging guidance to extend percutaneously to a location/region of interest. FIG. 1 also illustrates a second biodegradable lead 20a passing through hollow bore needle 200 for implantation. A representative ultrasound image of the area of the ulnar nerve is illustrates in the upper portion of FIG. 1. Implantable, biodegradable leads 20 include a biodegradable, electrically conductive core 22 having a distal end which is positioned in the vicinity of the tissue/nerve of interest (the ulnar nerve in the embodiment of FIG. 1). Implantable, biodegradable leads 20 further include a biodegradable insulator or insulating layer 24 around biodegradable conductive core 22 which begins at a point spaced from the distal end of conductive core 22.

The insets of FIG. 1 illustrate enlarged views of implantable, biodegradable lead 20 including single active non-insulated electrode at the distal tip of the lead and implantable, biodegradable leads 20′ and 20a′ (in a staggered octapolar arrangement) which include spaced insulating portions 24′, 24a′ at the distal end thereof to create a plurality of spaced electrodes 24″, 24a″ in which biodegradable conductive core 22′, 22a′ is exposed (to form a combination of a double tripole and a single, midline crossing tripole). Stimulation using multiple leads is, for example, described in Aló, K. M, New Trends in Neurmodulation for the Management of Neuropathic Pain, Neurosurgery, 50:4, 690-704 (2002), the disclosure of which is incorporated herein by reference.

Biodegradable leads or electrodes are previously unknown in the context of applying electrical signal to tissue in a treatment protocol (for example, in pain management). Leads hereof are, for example, designed to sustain relatively high-charge, long-term, and constant stimulation needed for such treatment protocols, including in pain management. In general, any treatment stimulation signal can be used in connection with the biodegradable leads hereof. Waveforms and other parameters for such signals are, for example, described in Head, J., et al., Waves of Pain Relief: A Systematic Review of Clinical Trials in Spinal Cord Stimulation Waveforms for the Treatment of Chronic Neuropathic Low Back and Leg Pain, World Neurosurgery, 131:264-274 (2019), Miller, J. P., et al., Parameters of Spinal Cord Stimulation and Their Role in Electrical Charge Delivery: A Review, Neuromodulation, 19: 373-384 (2016); and Levy, R., et al., Multicenter, Randomized, Double-Blind Study Protocol Using Human Spinal Cord Recording Comparing Safety, Efficacy, and Neurophysiological Responses Between Patients Being Treated With Evoked Compound Action Potential-Controlled Closed-Loop Spinal Cord Stimulation or Open-Loop Spinal Cord Stimulation (the Evoke Study), Neuromodulation, 22: 317-326 (2019), the disclosure of which are incorporated herein by reference.

in a number of embodiments, biodegradable conductive cores hereof have a conductivity of at least 10×10¹⁶ S m⁻¹. The conductive cores may, for example, include 1 to 100 strands of individual wires, each wire having a diameter in the range of 1 μm to 1000 μm. In a number of embodiments, the biodegradable conductive core has a Young's modulus in the range of 10 GPa to 400 GPa and a native degradation rate of 0.001 to 0.2 mm y⁻¹.

Biodegradable polymers for use in the biodegradable polymeric insulator layer hereof typically may, for example, be formed from monomers which are connected via biodegradable linkages or functional groups (for example, esters, anhydrides, orthro esters, amides etc.) and degrade in vivo to biocompatible degradation products. Synthetic biodegradable polymers suitable for use include, for example, polyurethanes, polyurethane copolymers, and poly(glycerol sebacate) (PGS). Many common degradable polymers are insulators. However, such polymers are too typically brittle and/or hydrolyze too quickly to be suitable for user herein. However, by including crosslinking in such polymers, one may obtain elastomeric materials that are mechanically more durable. Examples of such polymers include, for example, poly(diol citrates) or PGS. Implantable, biodegradable polymers such as polyurethane and polyurethane urea polymers and copolymers have been widely studied and characterized. A number of such polymers have been approved for implantation (for example, by the United States Food and Drug Administration or FDA).

Biodegradable polymers for use herein desirably exhibit biocompatibility, significant elastomeric properties, flexibility, toughness, resistance to water adsorption, and a relatively low dielectric constant. Polyurethanes, polyurethane ureas, and other polymers/copolymers may provide an excellent biocompatible, elastomeric (for example, capable of elastic deformations>300%) and low dielectric constant material for use herein. To ensure the strength and flexibility of the device, the targeted modulus and elasticity of the biodegradable polymeric insulators hereof may, for example, be at least 10 MPa and 100% strain, respectively. In a number of embodiments, the dielectric constant of the biodegradable polymeric insulators is between 1.01 and 3.5. The biodegradable polymeric insulators may, for example, exhibit a water absorption below 5% in a number of embodiments. The thickness of the biodegradable polymeric insulator is in the range of 1 μm to 500 μm in a number of embodiments hereof.

Biodegradable polyurethanes, for example, are hydrophobic and have tunable resorption rates and other physical properties. Moreover, polyurethanes are used in varieties of implantable medical devices including catheters, balloon pumps, artificial hearts, and pacemaker insulation. In a number of embodiments hereof, biodegradable polyurethanes (BPUs) were synthesized with degradable bonds (for example, ester bonds) in the backbone. Such polymers may be readily tuned for insulation properties and degradability to meet the requirement of stimulator leads hereof for various applications.

By optimizing the composition and thickness of the polymer insulation as well as the conductive core for a particular application, one may produce a stimulation lead which can provide effective treatment (for example, analgesic) stimulation for a given period while being harmlessly resorbed thereafter. Such a period of analgesic stimulation may lead to long lasting pain relief in many patients. As discussed further below, the rate of biodegradation of the conductive cores of biodegradable leads hereof may be further controlled via electrical signals from the electronic circuitry/pulse generators hereof. Additional stimulator leads can be injected if the pain comes back or moves to a different location. By replacing the components of the conventional designs with biodegradable materials, one may dramatically reduce complications seen with permanently implanted stimulators or temporary devices that have to be removed.

Referring again to FIG. 1, in the illustrated embodiment an exterior wire 50, which is in electrical connection with implantable lead 20 via a connector or a strain relief element 30 or which is an ex vivo portion of implantable lead 20, is connected to external (that is, ex vivo) electric circuitry 100 (sometimes referred to herein as a pulse generator) to generate an electrical signal. Although a portion of all of electronic circuitry 100 could be implanted, it is desirable to position electronic circuitry ex vivo to, for example, facilitate elimination of the requirement of a surgical procedure and the implantation of only biodegradable components of systems hereof. Electronic circuitry 100 may, for example, include a control system or controller as known in the tissue stimulation arts and the leads hereof may be activated using various control algorithms (for example, algorithms embodiment in software) as known in the tissue stimulation arts (for example, via periodic pulses of electrical energy). As illustrated schematically in FIG. 1, electronic circuitry 100 may, for example, include a power supply 110, a controller or control system (for example, including a processor or processors such as a microprocessor 120 and an associated memory system 130), a wired or wireless communication system 140 (for example, a BLUETOOTH and/or Wi-Fi antenna or transceiver), and an interface system 150 including one or more interfaces for input and/or output of data/information (for example, including a display 152, which may be a touch screen display). A sensor system 160 (illustrated schematically in FIG. 1) may include one or more sensors in operative connection with electronic circuitry (for example, via interface system 150). Such sensor(s) may, for example, measure data on position or posture of the patient and/or physiological/electrophysiological data to provide such data to electronic circuitry 100. Such data may, for example, be used to control (for example, via feedback control) electrical signals provide by electronic circuitry 100. Electronic circuitry 100 may, for example, be housed in a small housing unit 170. Housing unit 170 may, for example, be attached to the patient's skin via an appropriate adhesive as known in the medical arts.

As described above, multiple leads 20 may be implanted. One or more such leads 20 may be used to stimulate tissue. One or more such leads 20 may also be used in connection with sensor functionality. For example, one or more leads 20 may serve as a component of sensor system 160 to function as a recording electrode. In that regard, recent neuromodulation therapies not only provide output stimulation to the spinal cord or neural tissue but are also capable of serving as recording electrodes that deliver feedback to the electronic circuitry/pulse generator, which can therefore adjust stimulation accordingly. Systems that deliver fixed output with fixed parameters such as a set pulse width or a set frequency are referred to as open loop systems. Such systems with recording feedback are referred to as closed loop systems that can adjust stimulation by recording evoked compound action potentials (ECAPs), from the spinal cord or nerves and adjusting the stimulation accordingly. Such functionality is important because ECAPs are not static and may change based on patient position, respiratory cycle (expiration or inspiration), cardiac changes, etc. Without adjustment, a patient's stimulation may not be optimized at all times. Closed loop spinal cord stimulation has been presented, studied and tested clinically in the EVOKE trial described in Levy, R., et al., Multicenter, Randomized, Double-Blind Study Protocol Using Human Spinal Cord Recording Comparing Safety, Efficacy, and Neurophysiological Responses Between Patients Being Treated With Evoked Compound Action Potential—Controlled Closed-Loop Spinal Cord Stimulation or Open-Loop Spinal Cord Stimulation (the Evoke Study), Neuromodulation, 22: 317-326 (2019). In a number of embodiments hereof, electronic circuitry/pulse generator 100 is configured to adjust stimulation on the basis of feedback information from a biodegradable implanted lead 20 used as a recording electrode.

As described above, implanted lead(s) 20 will biodegrade over time but will remain functional for a predetermined and tunable/adjustable period of time (for example, from one to three months, or longer). System or device 10 thus has the potential to affect mid-term or long-term pain management. The effectiveness of the systems or devices hereof may even continue after lead degradation, for example, a result of a reduction in central sensitization of the spinal cord from the peripheral electrical stimulation.

In a number of embodiments, conductive core 22 of lead wire 20 is a biodegradable, electrically conductive metal or a biodegradable, electrically conductive metal alloy (for example, a binary or tertiary alloy). Biodegradable conductive core 22 may also include a biodegradable conductive polymer (such as polypyrrole, polythiophene and polyaniline and their derivatives) that has incorporated degradable linkages or include a composite material containing conducting polymer fillers and a biodegradable matrix. In general, there are two primary methods to create an extending, electrically conductive core including one or more conducting polymers. Degradable linkages may, for example, be introduced in the conjugated polymer. Alternatively, a composite can be formed which includes biodegradable conducting polymer fillers in a biodegradable matrix. Biodegradable insulating and conducting polymeric materials are, for example, discussed in Vivian, F. R., et al, Biodegradable Polymeric Materials in Degradable Electronic Devices, ACS Cent. Sci. 2018, 4, 337-348, the disclosure of which is incorporated herein by reference.

In a number of embodiments, biodegradable conductive core 22 includes magnesium (Mg) or a biodegradable and conductive alloy of magnesium. Typically, in the case of Mg, an alloy is required because of the relatively fast degradation rate of Mg. Metals that can be alloyed with magnesium (and/or other metals) for use herein include, for example, zinc (Zn), aluminum (Al), copper (Cu), Cerium (Ce), calcium (Ca), silver (Ag), thorium (Th), gadolinium (Gd), dysprosium (Dy), strontium (Sr), silicon (Si), manganese (Mn), zirconium (Zn), neodymium (Nd) and Yttrium (Y). Other biodegradable metals for use in biodegradable conductive cores hereof include iron (Fe), zinc, molybdenum, and tungsten (as well as alloys of such metals). Magnesium (Mg), zinc (Zn), and molybdenum (Mo) are, for example, used as biodegradable implants from bone screws to cardiovascular stents and physiological sensors. In a number of embodiments, biodegradable conductive core 22 includes zinc or an alloy of zinc. As described above, in a number of representative embodiments, biodegradable, polymeric insulator 24 includes a biodegradable polyurethane or polyurethane urea. Both conductive core 22 and insulator layer 24 may be designed to safely degrade/biodegrade, and the degradation rate thereof can be tailored by, for example, choice of biodegradable metal, alloying the biodegradable metal of conductive core 22 and adjusting the degradable linkages in the polymer backbone of insulator layer 24. The degradation products are biocompatible and safe and, in fact, may be beneficial as a result of analgesic, antimicrobial and/or neuroprotective effects of magnesium and/or other metals.

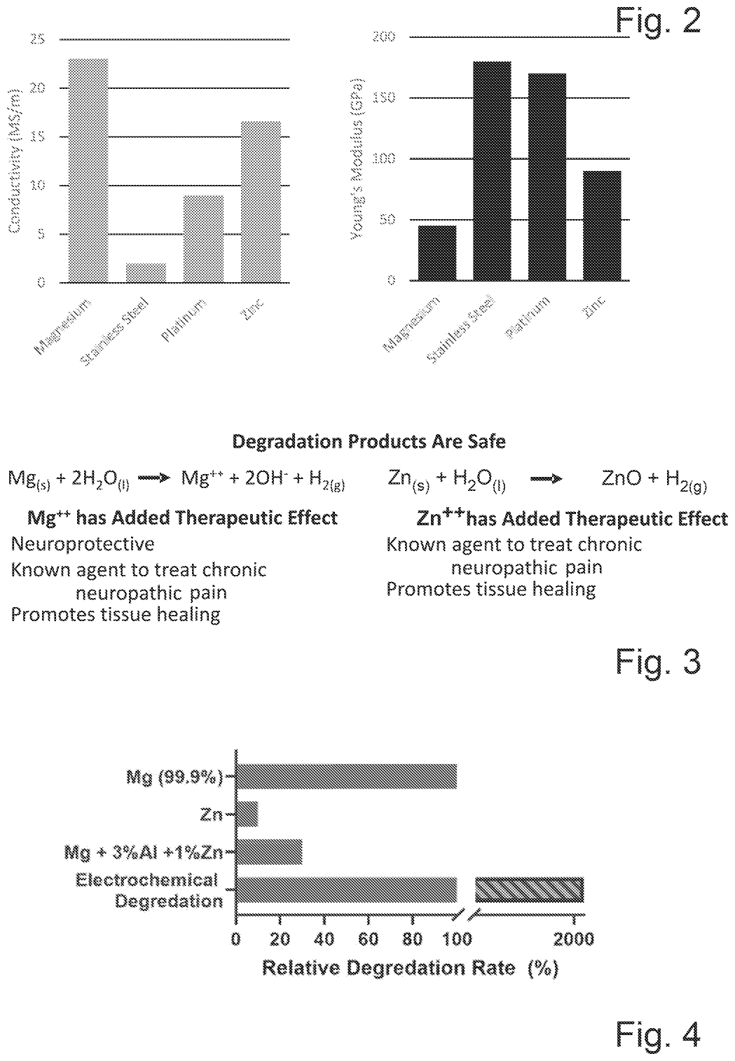

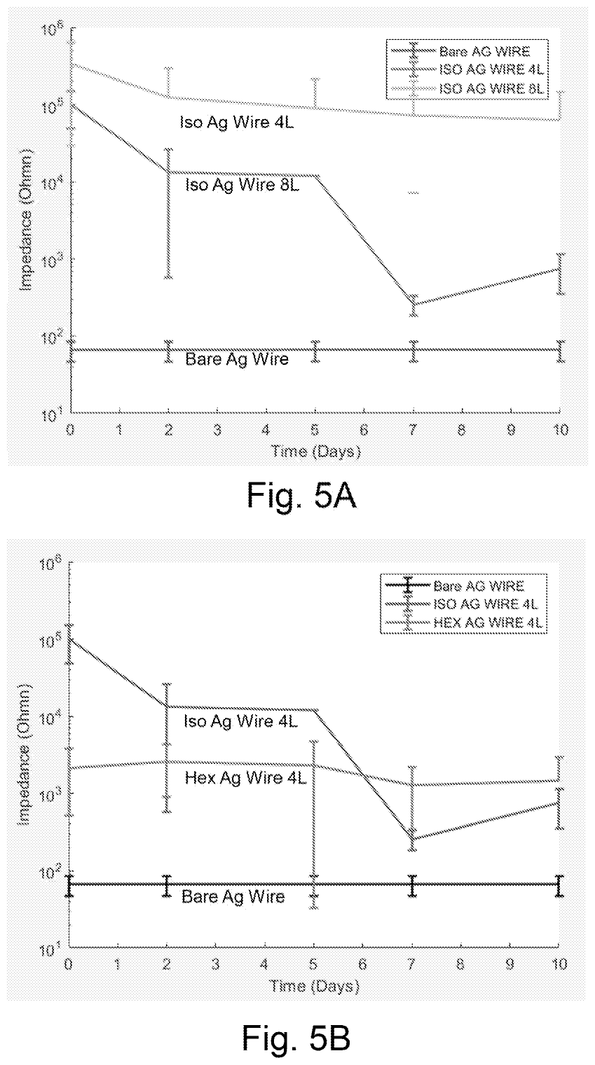

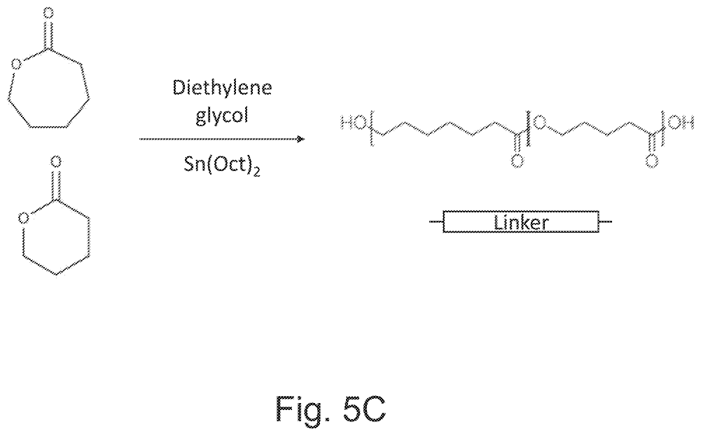

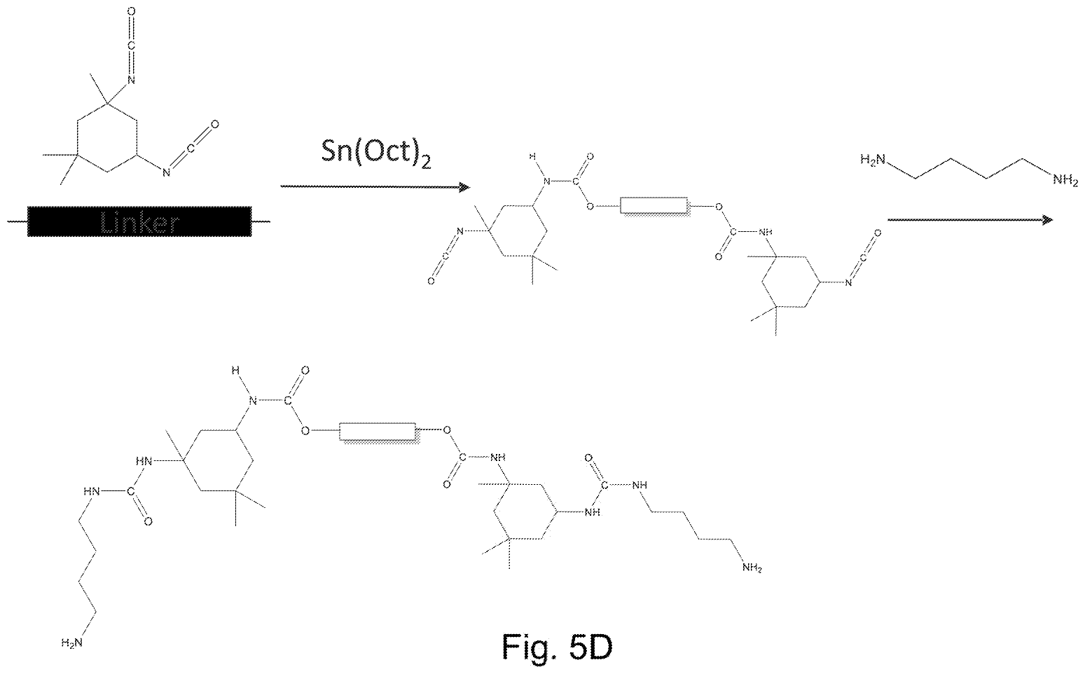

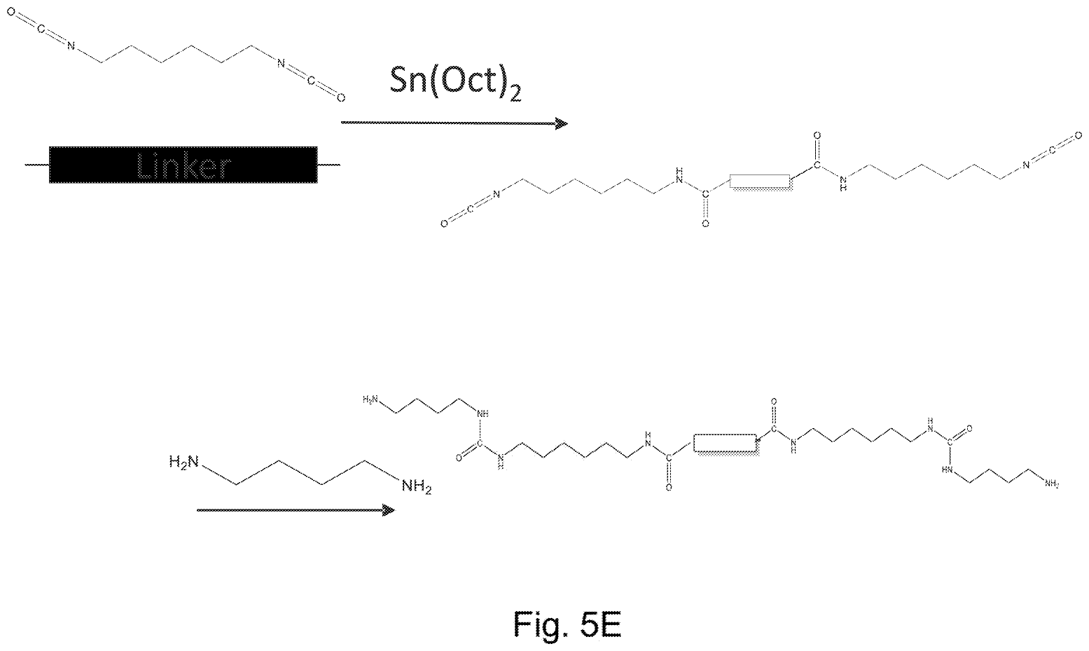

FIG. 2 illustrates a comparison of the conductivity and Young's modulus of magnesium, stainless steel, and platinum. As seen in FIG. 2, magnesium provides relatively high conductivity and low stiffness. The degradation chemistry of magnesium and the degradation products thereof are illustrated in FIG. 3. The Mg²⁺ cation is known to have added therapeutic effects including neuroprotection, beneficial effects on chronic neuropathic pain, and promotion of tissue healing. FIG. 4 demonstrates how the degradation rate of magnesium can be tuned via, for example, alloying. Some studies have used one or more layers of a conductive polymer such as PEDOT (Poly(3,4-ethylenedioxythiophene)) on a conductive core to control/slow degradation. Use of nonbiodegradable conductive polymer coatings such as PEDOT, however, may be undesirable in embodiments hereof. In general, it is undesirable to introduce a component to the leads hereof which is not biodegradable. In a number of embodiments, all components of the leads hereof are biodegradable. As described above, the degradation rate of a polyurethane-based polymer (or other polymer) may be controlled via the chemical composition (for example, choice or hydrolytic or other biodegradable linkages or copolymer composition). Figure SA illustrates degradation studies of an isophorone-based polyurethane polymer over time for four layers of polymer and eight layers of polymer on silver (Ag) wire wherein impedance at 1 kHz is set forth over time. FIG. 5B illustrates degradation studies of isophorone-based polyurethane polymer and hexamethylene-based polyurethan over time for four layers of polymer on silver (Ag) wire wherein impedance at 1 kHz is set forth over time. The synthesis and characterization of isophorone-based and hexamethylene-based polyurethanes for use as insulating layers on the leads hereof is set forth in FIGS. 5C through SE.

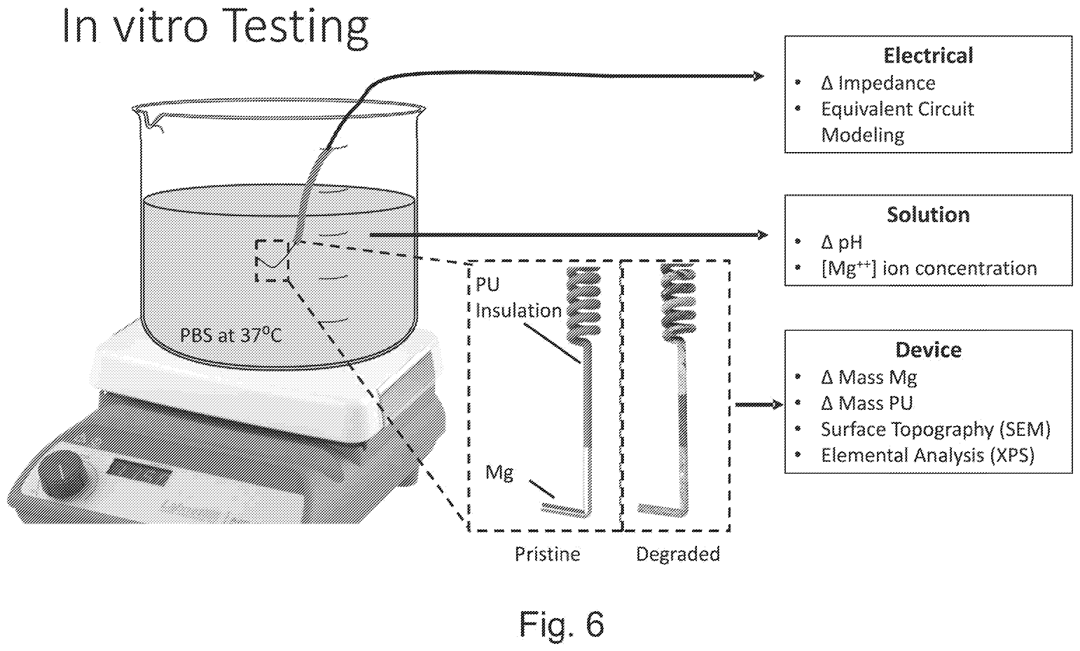

FIG. 6 illustrates an embodiment of a test system for in vitro assays to study, for example, the electrical and mechanical properties of a device or system hereof over time. The degradation profile and the identity and toxicity of degradation products may also be studied. The devices and system hereof may be further tested in vivo using, for example, a rat sciatic nerve model to, for example, compare material loss and tissue health between the degradable leads or electrode leads hereof and one or more non-degradable control leads or electrode leads.

As set forth above, conductive cores hereof formed from Mg degrade relatively quickly and may not be suitable for many electrical stimulation treatment protocols. Moreover rapid Mg degradation results in bubbling and an undesirable increase in pH as well as accumulation of hydrogen gas. Mg alloys may be preferable to Mg in such protocols.

In a number of representative studied embodiments of a biodegradable lead hereof, Zn was chosen as conductive core 22 because it exhibits intermediate degradation under physiological conditions and has non-toxic degradation products that may be beneficial for wound healing and pain management. A biodegradable polyurethane polymer or BPU was chosen as insulator layer 24 for the reasons discussed above, as well as the polymer's benign, natural, and biocompatible degradation products. The BPU used in the studies was synthesized and the electrode leads were fabricated using commercial Zn wires with an insulating layer of the synthesized BPU. The stimulation stability of the electrode lead was studied by applying waveforms used for tissue treatment in the form of PNS pain relief, with the duration of 60 days, which is equivalent to the length of implantation of the commercially available SPRINT® PNS system (available from SPR Therapeutics of Cleveland, Ohio).

As described above, biodegradable leads 20 hereof are energized by an external pulse generator/electronic circuitry 100. Once again, generally any stimulation treatment protocol as known in the stimulation arts can be used in connection with the biodegradable leads hereof. In a number of embodiments, the amplitude of the current of the stimulation signal(s) is in the range of 0.1 to 30 mA, the frequency is in the range of 10 Hz to 30 kHz, the pulse duration is in the range of 10 to 500 μsec, and the duty cycle is in the range of 1 to 100%. In a number of embodiments, the frequency is in the range of 10 Hz to 15 kHz or 10 Hz to 10 kHz, the pulse duration is in the range of 10 to 400 μsec or 10 to 200 μsec, and/or the duty cycle is in the range of 20 to 100% or 50 to 100%. As discussed further below, degradation of biodegradable cores hereof including metals and metal alloys may be enhanced by application of an anodic, oxidative electrical signal. In general, relatively long anodic pulses may be used to drive metal oxidation, and stimulation treatment protocols including such long anodic pulses may accelerate degradation. If a particular stimulation treatment protocol is found to cause undesirably rapid degradation, the treatment stimulation protocol may be altered and/or biodegradable lead 20 hereof may be altered as described herein.

In a number of embodiments, pulse generators hereof included custom-designed features to meet special requirements of the degradable devices/leads hereof. For example, in a number of embodiments, electronic circuitry 100 (for example, including or more integrated circuit (IC) chips) includes an active impedance measurement circuit which is configured to monitor the electrode changes and to adjust the stimulation current profile accordingly. Further, after the end of the pain relief stimulation, degradation on command (DOC) may be activated by applying anodic pulses to enhance or accelerate the degradation, resulting in the increases or complete resorption of the metal components within a given period (for example, within seven days). DOC shortens the degradation time to minimize unnecessary complications resulting from the implanted foreign body and/or to allow for rapid elimination of biodegradable lead 20 should any adverse event take place or if a second injection is needed. The electrical command profile for DOC may, for example, be optimized for maximum safety (for example, via control of amplitude, polarity, duration, etc. of DOC signals). Routine testing may, for example, be used to determine rates of degradation during three phases, under pain relief stimulation condition, under DOC stimulation, and after DOC without any electrical input.

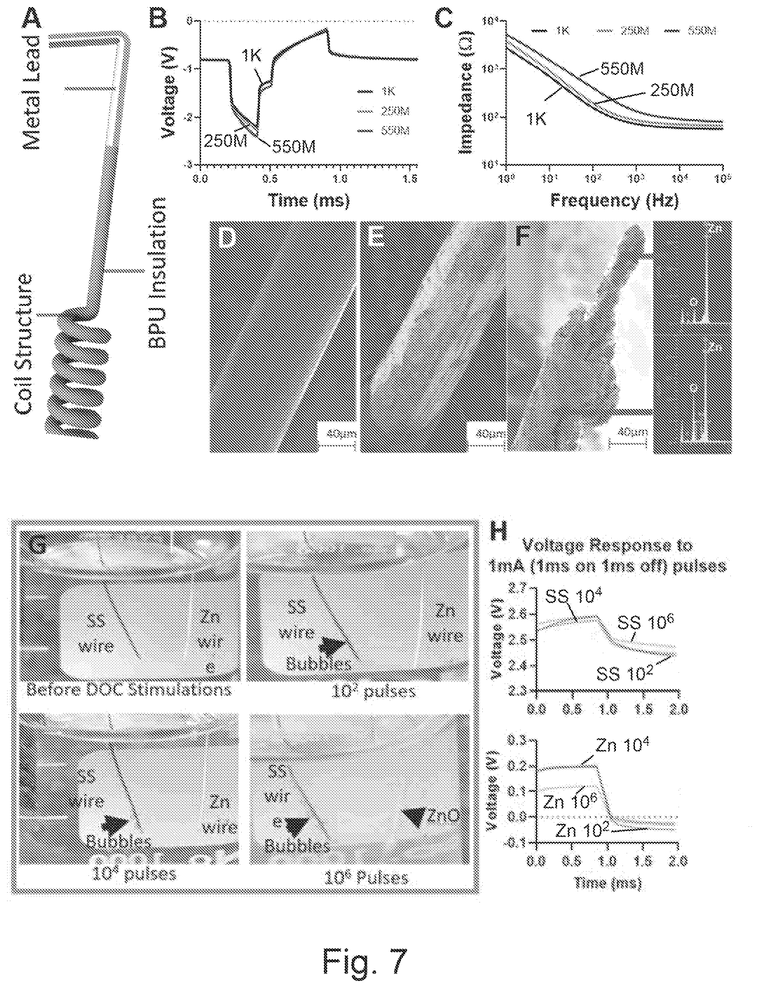

A schematic illustration of an embodiment of a Zn electrode lead hereof is provided in FIG. 7A. In several studies, the Zn electrode was based on commercially available Zn wire with a diameter of 100 μm. Single or multiple strands of wires are tightly coiled then insulated by dip-coating in a solution of BPU in tetrahydrofuran, leaving 1.5 cm of the Zn uncoiled and uninsulated as the electrode site. The tip of the Zn may be bent to form a hook, increasing the success of injection. If creating a hook at the distal end of the electrode does not prevent lead migration, one may, for example, include a barb in the design of the electrode or suture the lead at the exit point. Studies of such electrodes have shown that the Zn electrode lead is capable of over 550,000,000 stimulations, the equivalent of constant 100 Hz stimulation for 60 days (see FIGS. 7B-E). All in vitro electrical characterization and stimulation were performed in physiological saline. DOC trial experiments demonstrated that an anodic current of 0.5 mA for 20 hrs resulted in a rapid degradation of the Zn wire cores, with a near complete loss of the Zn evenly along the length of the electrodes (FIG. 7F). To test DOC, a monophasic anodic pulse train of 1 mA for 1 ms at a 50% duty cycle was initiated while monitoring for changes of the electrode voltage and for bubble formation. Using the 1 mA monophasic pulse train, it was shown that Zn can be safely dissolved without formation of gas bubbles or voltages exceeding 0.2V (FIGS. 7G and 7H). Once again, DOC parameters may be adjusted to balance speed and safety.

In the studies of FIG. 7B voltage excursions from a 20 μs-20 mA pulse were followed by a 40 μs 10 mA balancing pulse measured after 1K, 250M and 550M pulses. FIG. 7C illustrates a graph of impedance measured from Zn electrodes under the same conditions as FIG. 7B. FIGS. 7D and 7E illustrates SEM images of pristine Zn (FIG. 7D) and a Zn electrode after 250M stimulations. The insulation layer is visible at the base of FIG. 7E. In FIG. 7F, The Zn electrode was partially degraded by DOC to examine the rate and uniformity of the degradation. The Zn core is still present while the outside has been converted to a fragile oxide which brushed away. The degradation occurred evenly along the length of the wire. EDX studies revealed elevated oxygen in the corrosion layer (bottom) and elevated Zn in core (top). The photographs of FIG. 7G illustrate the progression of Zn degradation with DOC. The Zn electrode dissolved after 35 min without bubble formation. The SS electrode remained intact but generated bubbles. FIG. 7H illustrates studies of the voltage response of the SS and Zn electrode to anodic pulse train of 1 mA (1 ms on/off). There is a large difference in the maximum voltage. 0.2V (Zn) vs. 2.6V(SS).

As described above, the electronic circuitry/pulse generators hereof have enhanced degradation circuitry/functionality to initiate enhances/more rapid degradation to, for example, minimize unnecessary complications arising from the foreign body within the body, and allow for rapid elimination of the device/stimulation lead should any adverse event occur or if a replacement lead/electrode is needed. The electrically initiated degradation or degradation on command or DOC utilizes anodic current to oxidize the Zn electrode to Zn²⁺. The resulting ZnO can safely dissolve in body fluids. The feasibility of DOC was established by immersing stainless steel (SS) and Zn wires (1.5 cm long, 100 μm in diameter) in physiological saline, and stimulating them respectively using pulse trains of a 1 mA monophasic anodic waveform (1 ms on and 1 ms), for over 1000000 pulses. As set forth above, while bubbles were observed on the SS wire as a result of the water hydrolysis, the Zn electrode did not form bubbles. As the pulse number increased, the metallic looking Zn wire developed a white oxide and dissolved away after 1000000 pulses (approximately 35 min). The voltage transient measured with the Zn experiencing cathodic voltage far below the water hydrolysis and the oxidation potentials of common species in body fluids. Passive corrosion of Zn has been shown to be safe in vivo. Although there may have been some concern that electrically initiated, rapid degradation of biodegradable metals in DOC hereof might result in poor implant-tissue integration as a result of gas evolution and pH change, lack of observed bubbles, and maintenance of electrode potential at very low levels, eliminate such concerns.

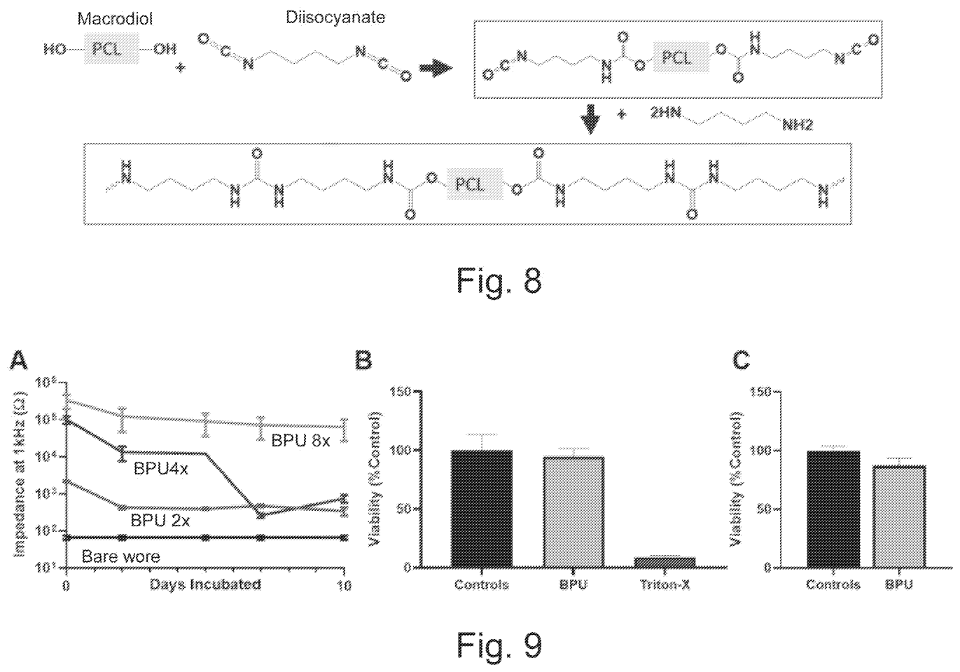

FIG. 8 illustrates an embodiment of a synthetic scheme for synthesis of the BPU of the insulating layer of the electrode leads hereof. The BPU was formed from of a biodegradable polycaprolactone diol (PCL), a diiosocyanate (1,4-diisocyanato butane in the illustrated embodiment), and 1,4-diaminobutane as the chain extender. To demonstrate the tailorable degradation of the BPU insulation, silver wires were dip-coated with BPU with different numbers of coats and then submerged in saline for 10 days at 37° C. Impedance measurements over time (FIG. 9A) indicated that the thickness of the insulation can be used to tune the quality of the insulation and rate of degradation. Toxicity assays were performed on the BPU using both direct contact and elution assays. No observable toxicity was found (see FIGS. 9B and 9C). More comprehensive toxicity testing may be conducted following, for example, ISO 10933 standards (parts 5, 13, 15, and 16) on the polymer, the metal and their degradation products.

Control of the degradation rate of the BPU may, for example, be achieved by adjusting the composition thereof. Referring to the synthetic scheme of FIG. 8, the length of the biodegradable (for example, PCL) segment therein may be adjusted, which can be controlled by altering the ratio of diethylene glycol to caprolactone during the synthesis of the macrodiol. As discussed above, a representative BPU used in a number of studies hereof was formed by reacting the PCL with the diisocyanate followed by the addition of a chain extender. Often, aliphatic diisocyanates are used in biocompatible polymers. Example of such aliphatic diisocyanate include, but are not limited to, 1,6-hexamethylene diisocyanate, 1,4-diisocyanato butane, L-lysine diisocyanate, isophorone diisocyanate, 1,4-diisocyanato 2-methyl butane, 2,3-diisocyanato 2,3-dimethyl butane, 1,4-di(1propoxy-3-diisocyanate, 1,4-diisocyanato 2-butene, 1,10-diisocyanato decane, ethylene diisocyanate, 2,5 bis(2-isocyanato ethyl) furan, 1,6-diisocyanato 2,5-diethyl hexane, 1,6-diisocyanato 3-methoxy hexane, 1,5 diisocyanato pentane, 1,12-dodecamethylene diisocyanate, 2 methyl-2,4 diisocyanato pentane, 2,2 dimethyl-1,5 diisocyanato pentane, ethyl phosphonyl diisocyanate, 2,2,4-trimethyl-1,6-hexamethylene diisocyanate, 4,4′-dicyclohexylmethane diisocyanate (H₁₂MDI), trans 1,4-cyclohexane diisocyanate, m-tetramethylxylylene diisocyanate, m-isopropenyldimethylbenzyl, “dimeryl” diisocyanate derived from dimerized linoleic acid, xylylene diisocyanate, and 1,1,6,6-tetrahydroperfluorohexamethylene diisocyanate. Biodegradable segments for use in the biodegradable polymers hereof may, for example, include poly epsilon caprolactone, poly gamma caprolactone, poly gamma butyrolactone, poly gamma valerolactone, poly delta valerolactone, poly lactic acid, poly glycolic acid, poly sebacic acid, poly(glycerol sebacate), and any copolymers thereof. Representative chain extenders include but are not limited to putrescine (1,4 diamino butane), diaminohexane (1,6), spermine, spermidine, lysine, ornithine, and isophorone diamine.

The representative Zn electrode leads hereof are capable of providing effective electrical stimulation for at least 60 days under simulated physiological conditions. Following stimulation, DOC may be used to cause a rapid and uniform destruction of the Zn wire. The DOC protocol may be optimized to minimize or prevent cellular damage. As described above, the thickness of the BPU insulation as well as the PCL length may be tailored such that it is stable during the stimulation period and degrades thereafter. If tuning thickness and PCL length is insufficient to achieve optimum BPU degradation profile for a particular application, alterations may readily be made to the composition of the polymer backbone by, for example, copolymerizing the polycaprolactone with other polyesters such as valerolactone or by replacing the soft segment with a different degradable polyester such as polylactic acid or polyanhydrides. If the incorporation of some of these polyesters excessively increases the brittleness of the insulating layer, one or more elastomeric component(s) (for example, polygycerosebate) may be incorporated.

If a DOC is determined to be too harsh for a particular application, the intensity of duty cycle of the DOC signal may, for example, be reduced while, for example, extending the DOC period. Even without the DOC, the biodegradable leads hereof still passively degrade which addresses the disadvantages of PNS using permanent, non-biodegradable materials.

The parameters of the DOC may be readily determined or optimized to effect degradation of a particular biodegradable conductive core material in a desired time period through literature and/or routine experimentation as described herein. Such parameters may be readily determined to minimize or eliminate damage to tissues. Degradation products produced by the DOC signal are biocompatible. In a number of embodiments, potentials at the electrode surface do not exceed 0.4V. The DOC waveform may, for example, be monophasic (anodic only) or biphasic (with more anodic charges per phase than cathodic) and with long pulse duration. In a number of embodiments, the pulse width of the anodic pulse of the DOC signal is between 1 ms to multiple seconds. The frequency is between 1 kHz to 0.1 Hz in a number of embodiments. In a number of embodiment, the amplitude of the DOC signal is between 0.1 mA to 20 mA or it could be direct current (DC). In other cases, direct current may be applied for extended periods (for example, hours) with or without intermittent resting periods.

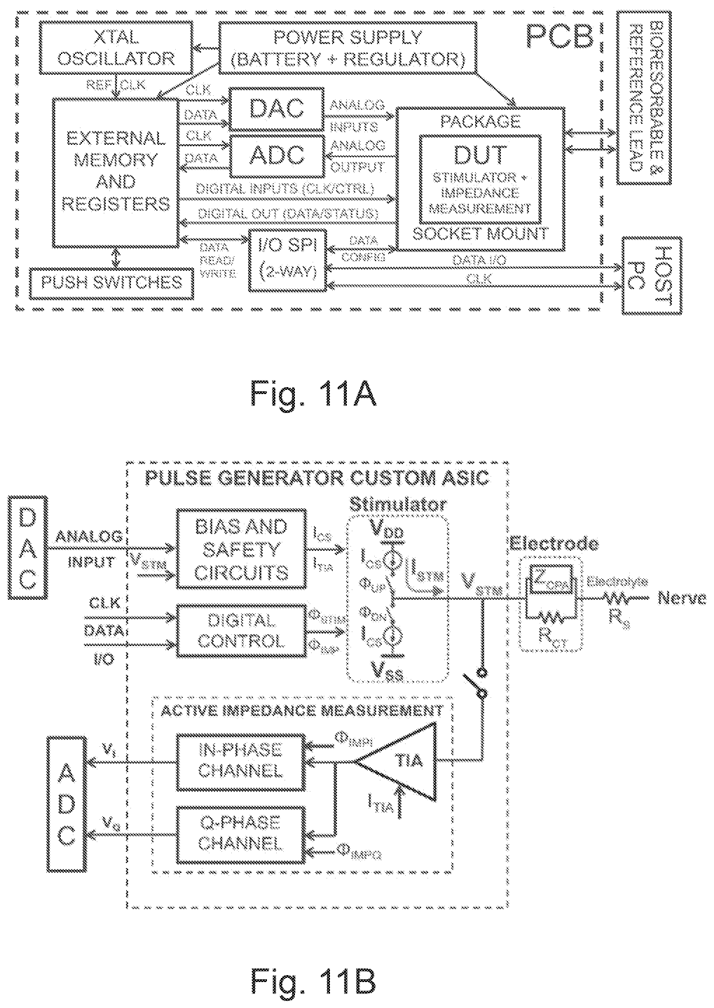

As described above, in a number of embodiments, external pulse generator 100 hereof provides one or more of the following three functions: 1) it provides current controlled stimulation with programmable parameters, 2) it uses an active impedance measurement circuit to adjust the stimulation profile and achieve maximum stimulation efficiency even when the electrode/tissue impedance changes, and 3) it enables the delivery of DOC pulses. For in vivo testing, a miniature pulse generator may be used that can be strapped to a rat's back. Such a backpack pulse generator includes a printed circuit board (PCB) that integrates the battery and a crystal oscillator with the pulse generator module, an external memory to collect and store long-term data and an interface such as a 2-way Serial Peripheral Interface (SPI) block to communicate to a Host PC (see FIG. 11A). The pulse generator may, for example, be placed inside of a 3D printed housing designed to be harnessed to the rat's back. In a number of embodiments, a conductive (for example, titanium) plate is included at the base of the housing, which is designed to be in contact with the skin to operate as a counter electrode. The backpack pulse generator may, for example, be connected to the Host PC only when it is required to read stored data or use the impedance measured to adjust the profile using a chip-in-loop algorithm.

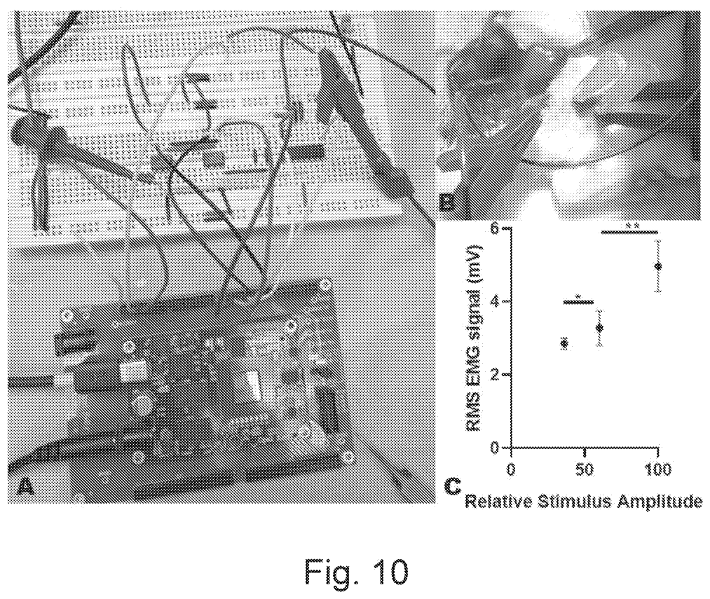

A breadboard model of pulse generator 100 has been validated in vitro and in vivo. The generated waveform has been validated with an oscilloscope, confirming the ability of the device to produce monophasic capacitor coupled pulses in addition to the DOC stimulus. The breadboard model was then validated in a rat model by stimulating the sciatic nerve to evoke muscle activity with a Zn stimulating electrode (see FIG. 10).

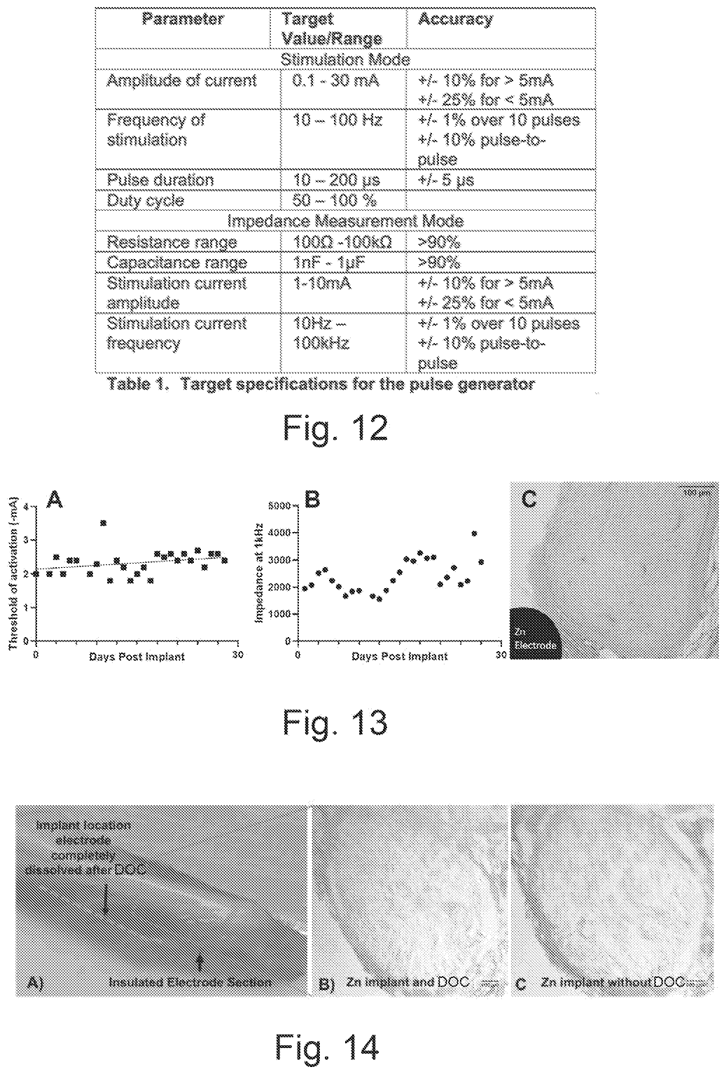

An embodiment of a fully integrated chip (IC) based on the tested breadboard prototype is shown in FIG. 11B. The IC provides the analgesic stimulation and the DOC waveform. The IC may, for example, be provided with closed loop feedback which adjusts the stimulation and DOC waveforms based on live impedance measurements. The block diagram of an embodiment of the architecture is illustrated in FIG. 11B and representative specifications therefore are set forth in Table 1 of FIG. 12. The parameters of Table 1 are representative for peripheral nerve stimulation. In the case of spinal cord stimulation, for example, higher frequencies may be used.

In a number of embodiments, external pulse generator/electronic circuitry 100 uses a current stimulator with programmable amplitude, frequency and pulse duration. The stimulation modes may, for example, include monophasic and biphasic capacitor coupled and biphasic asymmetric pulses which replicate clinically applied stimulations. To realize high accuracy current control, a high-resolution digital-analog converter (>8 bits) may be used. In DOC mode, the degradation of the stimulating electrode is accelerated by passing anodic current pulses with a high duty cycle. A safety measure may be implemented at a cutoff voltage at V_{STM}, as shown in FIG. 11B.

In a number of embodiments of an impedance measurement and calibration mode, the stimulation current is applied on the tissue and the complex voltage developed across the tissue interface is measured using the front-end stage of the chip. The real and imaginary parts of the voltage across the impedance may be separately measured using quadrature demodulation in two channels, In-phase (I) and Quadrature (Q)) channels, respectively. The voltages can be used to determine the resistance and capacitance associated with each dominant pole. An error correction algorithm may be used to reduce the error in measuring the output voltages that occurs as a result of the distortion introduced by higher order odd harmonies in the square wave input current.

The systems hereof were tested in a rat neuropathic pain model. Using a rat model, one may gather important information about the host tissue reactions to the device, demonstrate long term in vivo biocompatibility, and validate the degradation timeline. In vivo experiments may also be used to examine the mechanical and electrochemical performance in freely behaving animals. Finally, such studies may be used to demonstrate the ability of the device to relieve pain for the 60-day intended application period.

The bioresorbable leads hereof may, for example, be placed via ultrasound-guided injection near the rat sciatic nerve. This location allows observation of the effects of the device, the electrical stimulation, and the degradation on multiple tissues of interest. Further, the analgesic effect of the bioresorbable electrode over a defined treatment period (for example, 60 days) and subsequent resorption may be studied. Electrochemical measurements may be performed periodically (for example, daily or weekly) to monitor the changes in the electrode-tissue interface and validate the pulse generator functions. One may, for example, monitor the activation threshold of the hind limb, which examines if the electrode has fractured, moved after implantation, or otherwise becomes incapable of performing stimulation. The safety of the device may, for example, be evaluated postmortem by histological examination of the tissues.

A number of animal experiments to assess the viability of Zn-based electrode leads described above as a stimulating electrode have been performed in rats. In such studies (see FIGS. 13A through 13C), BPU insulated Zn electrodes were implanted next to the sciatic nerve and routed subcutaneously to a headcap. Asymmetric stimulations (−1.5 mA for 100 μs and a 0.75 mA for 200 μs) were performed for 30 days, delivering double the daily pulses to simulate 60 days. Muscle activation potential was recorded over the study and was found to be stable. The impedance was recorded over the study as illustrated in FIG. 13B. As illustrated in FIGS. 14A through 14C, histology shows no abnormalities in the nerve or muscle after a DOC procedure to degrade the Zn electrode.

The safety of the DOC methodology hereof was also established in vivo. In a number of studies, two Zn electrodes were fabricated by dip-coating 100 μm Zn wires with degradable polyurethane as described above, leaving 1.5 cm of the Zn exposed as charge injection site. A Sprague Dawley rat was anesthetized and received bilateral implants. Each electrode was placed inside an 18-gauge sterile needle which was inserted near the sciatic nerve. Gentle pressure was applied to the end of the needle, which was then withdrawn, leaving the electrode in place. One electrode was used for DOC while the other served as a passive control. A monophasic stimulus (1 mA for 1 ms followed by a 4 ms rest period) was applied to the electrode undergoing DOC (using a needle on the back as the counter) for 200 min. Five days after the DOC, the animal was sacrificed and the leg tissue on both sides were dissected. The uninsulated portion of the electrode has completely degraded in the electrode which underwent DOC, with no observable damage to the surrounding tissue or the target nerve. The control implant which did not undergo DOC showed no sign of degradation or tissue damage.

The devices, system, methods, and compositions hereof thus provide a minimally invasive avenue for nerve stimulation. The biodegradable leads hereof, when used in a nerve stimulator, will help chronic pain patients to improve their pain scores, improve their functional status, and either stop or wean opioids during the continued opioid epidemic. The devices, systems, methods, and compositions hereof provide a significant improvements over currently available nerve stimulators including, but not limited to, a significantly lower cost (associated with a minimally invasive implantation technique) and an improved safety profile since surgery and permanent implantations are avoided.

As described above, the biodegradable stimulator hereof may be inserted in an outpatient setting via, for example, a hollow-bore needle. After removal of the needle, only the biodegradable stimulator lead remains in place over the target nerve. The biodegradable stimulator lead or leads are attached to external pulse generator/electronic circuitry providing a controllable power source. The external electronic circuitry may, for example, be attached to the patient via an adhesive sticker on the skin. The implantation procedure does not require anesthesia or operating room time. The stimulator provides controlled electrical energy stimulation of the nerve (such as saphenous nerve of the leg or median nerve of the forearm) for a predefined amount of time until the stimulator significantly biodegrades. The pulse generator and electronic circuitry remain outside the body affixed to the skin and may, for example, be directly attached to the proximal end of the stimulator lead. The biodegradable devices or systems hereof solve many of the problems associated with current nerve stimulator devices such as a need for implantation surgery and the need for revision surgery for lead fractures, breakdowns, or migration. Furthermore, when a patient's pain improves or if a patient decides the device is no longer helping, the biodegradable device hereof will not remain inside the body indefinitely (even absent removal surgery). Further, the degradation products of the device may provide a therapeutic solution around the nerve.

### Experimental

Polymer characterization. After purification by precipitation, biodegradable polymers (for example, BPU) hereof may be subjected to NMR to, for example, determine the content and purity of a sample as well as its molecular structure. Elasticity and Young's modulus may be studied according to ASTM D412. The ability of the BPU to serve as an effective insulation may be studied through electrochemical impedance spectroscopy (EIS) monitoring of BPU insulated inert metal wires submerged in saline under physiological conditions for at least two months. The effects of oxidative degradation may be studied through the addition of H₂O₂. EIS and charge injection limit (CIL) measurements may be performed regularly throughout the 60-day stimulation period described above to examine the Zn-electrolyte interface.

Characterization of Degradation. Degradation is characterized during three phases as follows: 1) 60-day stimulation period, 2) DOC, and 3) passive degradation after the DOC. Periodically, the is sampled and solution pH is determined. Zn ion (or other metal ion) release may be determined via inductively coupled plasma mass spectroscopy (ICP-MS) following a protocol published in Catt, K.; Li, H.; Hoang, V.; Beard, R.; Cui, X. T., Self-powered therapeutic release from conducting polymer/graphene oxide films on magnesium. Nanomedicine: Nanotechnology, Biology and Medicine 2018, 14 (7), 2495-2503, the disclosure of which is incorporated herein by reference. In addition, the mass change of the leads may be characterized. The lead may be examine at different stages using, for example, scanning electron microscopy (SEM) and Energy Dispersive X-Ray Analysis (EDX) to track the physical and chemical changes which occur during stimulation, DOC, and aging studies.

The rate of degradation of the biodegradable cores hereof (for example, Mg, Zn etc.) is largely dependent on interactions with water and may be different in vivo than in vitro. Based upon the presence and extent of such difference, one may readily adjust the device size, geometry, insulation composition or exchange the core (for example, from Zn to the more active metal Mg or less active Mo).

Characterization of Degradation Products. The materials and their degradation products are subjected to in vitro toxicity assessments following ISO 10993 standards. It is desirable that the in vivo devices hereof be sterilized with ethylene oxide (ETO). To eliminate the potential effect of ETO on BPU degradation, all in vitro degradation samples are ETO sterilized.

Pulse generator validation. External pulse generators 100 hereof may, for example, be validated in three stages. A first stage may include stimulating and measuring a known impedance that models the bioresorbable lead and the underlying tissue, using ideal and known values of resistors I and capacitors 1. Such a validation stage may be used to establish the efficiency, accuracy and range of stimulation and impedance measurement. A second stage may include stimulating and measuring in-vitro where the proposed bioresorbable lead is, for example, placed in saline. In a third and final stage, pulse generator 100 may be validated in vivo with tissue stimulation and impedance measurement. Specifications for the prototype are captured carefully, accounting for margin for error and variation. For example, range of stimulation current (amplitude, time duration) can be programmed with a wider range for maximum coverage and flexibility. Proper planning and error mitigation circuits may be built into the design. Charge balancing may be accomplished so that there is no net accumulated (except for the DOC protocol).

Animal studies. To induce neuropathic pain, the sciatic nerve may be partially ligated with a surgical suture. The wound is then closed, and the animal allowed to heal for two weeks prior to insertion of the nerve stimulator. In a number of studies, a plurality of groups of rats (for example, 9 groups of rats (n=12, total 108)). The sample size may be determined with a power analysis for a continuous endpoint measurement, with an alpha value of 0.05 and a power of 80% and is split (1:1) between genders. In a number of study designs, a control group receives the nerve ligation surgery but no stimulator. Six groups of animals receive the ligation surgery and are implanted with the Zn electrode then sacrificed at 67, 120, or 180 days to assess the histological responses and bioresorption of the device. 3 of these 6 groups are used to examine the effect and extent of DOC. Finally, two groups are implanted with permanent stainless steel (SS) devices similar to the clinical standard of care. Electrodes may, for example, be injected with a modified Seldinger technique. See, for example, Yoon, H. K.; Hur, M.; Cho, H.; Jeong, Y. H.; Lee, H. J.; Yang, S. M.; Kim, W. H., Effects of practitioner's experience on the clinical performance of ultrasound-guided central venous catheterization: a randomized trial. Sci Rep 2021, 11 (1), 6726. First, an 18-gauge hollow bore needle with a stylet us inserted from the back to the sciatic nerve guided by a Vevo 3100 ultrasound with MX550 transducer. The stylet is removed and replaced with a stimulating probe. The proximity to the nerve may be confirmed by evoking gastrocnemius activity. Once the proper location is confirmed, the probe is removed, and the Zn electrode inserted. Gentle counter-pressure is applied while removing the outer cannula. A 20 cm lead that exits the skin directly below the location of the pulse generator is connected to the pulse generator via an insulation penetrating clip. Next, a 100 Hz 10 μs charge balanced stimulus is applied, for example, for 24 h a day at 1.5 mA, which is the duration and frequency of which are based on typical PNS stimulation for pain. Periodic (for example, weekly) measurements of the muscle activation current may be made to confirm the location of the device has not shifted. The sensation of pain may be assessed with von Frey hairs and paw withdrawal, measured once before sciatic nerve ligation (SNL), 1 and 2 weeks after SNL and then weekly after injection. Animals receiving effective pain relief stimulation require greater force to elicit paw withdraw than control rats. Rats are perfused at predetermined endpoints. Samples of muscle and nerve tissue by the implant are taken for histological staining and testing as detailed in ISO 10993. In vivo studies provide increased understanding of the device degradation profile with and without DOC based on the in vivo electrochemical and electrophysiological measurement as well as the explant and histological analysis at different time points over a test period. Periodically, (for example, once a week) the pulse generator is connected to a PC to, for example, read on-chip EIS measurements and to adjust the stimulation pulses if necessary. The leads may also be connected to a potentiostat to validate the pulse generator's function.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. An implantable system, comprising:
at least one implantable lead comprising a biodegradable conductive core, a biodegradable polymeric insulator encompassing at least a portion of a length of the biodegradable conductive core, and at least one electrode on a distal end thereof, and
electronic circuitry operatively connectible to the at least one implantable lead which is configured to provide a controlled electrical signal via the at least one electrode of the at least one implantable lead to tissue to effect treatment.

2. The system of claim 1 wherein the controlled electrical signal comprises pulses of electrical energy.

3. The system of claim 1 wherein the biodegradable conductive core comprises at least one of a biodegradable metal, a biodegradable metal alloy or a biodegradable conductive polymer.

4. The system of claim 3 wherein the biodegradable conductive core comprises a biodegradable metal or a biodegradable metal alloy.

5. The system of claim 4 wherein the biodegradable metal or the biodegradable metal alloy comprises magnesium, a magnesium alloy, iron, an iron alloy, zinc, a zinc alloy, molybdenum, a molybdenum alloy, zirconium, a zirconium alloy, calcium, or a calcium alloy.

6. The system of claim 5 wherein the magnesium alloy comprises at least one of zinc, aluminum, copper, cerium, calcium, silver, thorium, gadolinium, dysprosium, strontium, silicon, manganese, zirconium, neodymium or yttrium.

7. The system of claim 3 wherein the biodegradable polymeric insulator comprises a biodegradable polyurethane or polyurethane urea polymer or copolymer.

8. The system of claim 3 wherein the electronic circuitry is configured to be positioned ex vivo when placed in operative connection with the at least one implantable lead and the implantable lead is configured to be implanted percutaneously.

9. The system of claim 3 wherein the composition of the biodegradable conductive core is formulated to provide a predetermined degradation profile over time.

10. The system of claim 3 wherein the composition of the biodegradable polymeric insulator is formulated to provide a predetermined degradation profile over time.

11. The system of claim 3 wherein the system comprises a plurality of implantable biodegradable leads.

12. The system of claim 11 wherein at least one of plurality of implantable biodegradable leads functions as a recording electrode and the electronic circuitry is configured to adjust the controlled electrical signal on the basis of feedback information from the recording electrode.

13. The system of claim 5 wherein the biodegradable conductive core comprises zinc.

14. The system of claim 1 wherein the electronic circuitry is further configured to measure impedance at the interface of the tissue and the at least one implantable lead via which the controlled electrical signal is provided and to adjust the controlled electrical signal on the basis of the measured impedance.

15. The system of claim 1 wherein the electronic circuitry is further configured to transmit a degradation acceleration electrical signal to the least one implantable lead to increase a rate of degradation thereof.

16. The system of claim 15 wherein the electronic circuitry is further configured to measure impedance at the interface of the tissues and the at least one implantable lead via which the controlled electrical signal is provided and to adjust the controlled electrical signal on the basis of the measured impedance.

17. The system of claim 15 wherein the degradation acceleration electrical signal converts the biodegradable conductive core to a form more readily resorbed in vivo.

18. The system of claim 15 wherein the biodegradable conductive core comprises a metal or a metal alloy and the degradation acceleration electrical signal oxidizes the metal or the metal alloy.

19. A method of transmitting electrical signals to in vivo tissue for treatment, comprising:
implanting at least one implantable lead comprising a biodegradable conductive core, a biodegradable polymeric insulator encompassing at least a portion of a length of the biodegradable conductive core, and at least one electrode on a distal end thereof, and
applying a controlled electrical signal to the tissue via the at least one electrode of the at least one implantable lead via electronic circuitry operatively connected to the at least one implantable lead.

20.-32. (canceled)

33. The method of claim 19 wherein the controlled electrical signal comprises a plurality of pulses of electrical energy which are applied to the tissue via the at least one electrode of the at least one implantable lead and the electrical signal is pulsed for pain therapy electrical stimulation.

34.-38. (canceled)

39. The system of claim 1 wherein the at least one implantable lead consists essentially of:
the biodegradable conductive core formed of a metal or a metal alloy, and
the biodegradable polymeric insulator which is positioned directly adjacent to and encompasses at least a portion of a length of the biodegradable conductive core.

40.-56. (canceled)
